(19) United States
(12) Patent Application Publication
(10) Pub. No.: US 2008/0051646 A1
(43) Pub. Date: Feb. 28, 2008

(54) Probe for cellular oxygen

(76) Inventors: Dmitri Boris Papkovsky, County Cork (IE); Tomas O'Riordan, Cork (IE); Gelii Ponomarev, Moscow (RU)

Correspondence Address: JACOBSON HOLMAN PLLC, 400 SEVENTH STREET N.W., SUITE 600, WASHINGTON, DC, 20004, US

(21) Appl. No.: 11/878,343

(22) Filed: Jul. 24, 2007

(30) Foreign Application Priority Data
Jul. 24, 2006 (IE) IE2006/0544

(51) Int. Cl. A61B 5/1459

(52) U.S. Cl. 600/329

(57) ABSTRACT

A probe for sensing and imaging intracellular oxygen comprises an oxygen-sensitive fluorescent or phosphorescent dye combined with a hydrophilic macromolecular carrier and a cell loading agent. A method for sensing cellular oxygen using the probe is also described.

### INTRODUCTION

The invention relates to a probe for detecting oxygen.

Molecular oxygen (O₂) is the key metabolite in aerobic cells and organisms which is continuously consumed and/or released by live cells. Analysis of cellular oxygen consumption can provide valuable information about the general status, metabolic activity, viability, disease state of the cell or organism, their physiological responses, for example, to a drug, toxicant, effector, environmental stress, or other stimuli. Therefore, measurement of cellular oxygen is a vital analytical technique for many areas of biomedical and life science research.

Biological oxygen consumption can be quantified by measuring pressure change in the headspace of samples placed in closed test-vials (Eden and Sullivan 1992). Electrochemical oxygen detection using Clark-type electrodes has been used extensively, but its invasive and consumptive nature is a serious drawback. More recently, optical schemes based on the quenching by molecular oxygen of long decay fluorescent and phosphorescent dyes such as metalloporphyrins and ruthenium(II) complexes have been developed (Papkovsky 2004). Quantitation of oxygen by luminescence quenching has a number of advantages and attractive features.

Optical oxygen sensors/probes usually comprise an oxygen-sensitive dye in an appropriate quenching medium which is exposed to the sample. U.S. Pat. No. 4,003,707 (Lubbers and Optiz 1977) and U.S. Pat. No. 4,810,655 (Khalil, Gouterman et al. 1989) describe systems which employ solid-state oxygen-sensitive materials based on fluorescent pyrene butyrate and phosphorescent palladium(II)- and platinum(II)-porphyrins, respectively. Oxygen sensitive materials based on fluorescent ruthenium dyes embedded in polymers such as silicon rubber (Bacon and Demas 1991) and Pt- and Pd-complexes of porphyrin-ketones in polystyrene and other polymers (U.S. Pat. No. 5,718,842; Papkovsky and Ponomarev 1998) have also been described. Such solid-state oxygen sensors are usually prepared in the form of a coating or a membrane permeable to oxygen which is brought into contact with a test sample where oxygen concentration is to be determined.

Water-soluble fluorescence and phosphorescence based oxygen-sensitive materials have also been described. For example, Rumsey (Rumsey, Vanderkooi et al. 1988) described a method and apparatus for imaging of oxygen distribution in tissue using phosphorescent Pd-porphyrins. Similarly Vinogradov (Vinogradov, Lo et al. 1996) described water-soluble non-covalent complexes of Pd(II)-tetrabenzoporphyrins with serum albumin as extracellular oxygen probes for imaging tissue oxygenation. These probes are suitable for fluorescence lifetime-based detection of oxygen, however they have an undefined chemical composition, and there is the possibility of the dye binding to cells and other sample components, in addition to self-quenching of the dye and potential phototoxic action on cells. Pd-tetrakis-(4-carboxyphenyl)porphine and Pd-tetrakis-(4-carboxyphenyl)benzoporphine modified with multiple polyethyleneglycol (PEG) and branched polyglutamate chains have been suggested as soluble oxygen probes (Vinogradov and Wilson 1998). Wilson and Vinogradov (2002) describe the use of such a soluble oxygen probe in cell-respirometric assays and drug screening applications. These probes emit (phosphoresce) at above 700 nm and can be measured using CCD cameras and semiconductor detectors. Again, these probes were designed for extracellular use, for measurement of oxygen consumption in biological samples and/or oxygen distribution in large objects such as live tissues and organs.

Using the optical oxygen sensing approach, a number of formats for measuring biological oxygen consumption in microtitre plates on a fluorescent plate reader using phosphorescent solid-state oxygen sensors (O'Riordan, Buckley et al. 2000; John, Klimant et al. 2003) and water-soluble probes (Hynes, Floyd et al. 2003) have been developed. These systems allow measurement of oxygen consumption in biological samples non-invasively, on a micro-scale and with high sample throughput. They have been used with bacteria (John, Klimant et al. 2003), isolated mitochondria (Hynes, Marroquin et al. 2006), cell lines (Hynes, Hill et al. 2005) and whole organisms (O'Mahony, O'Donovan et al. 2005). These methods rely on the measurement of extracellular oxygen in relatively large samples and space, i.e. global rather than local oxygen gradients and they all use extracellular oxygen probes/sensors.

Sensing of intracellular oxygen can provide a more detailed insight into cellular function and metabolism, and cellular responses to various stimuli. Electrochemical microsensors for intracellular oxygen measurement have been described, but they are consumptive, discrete and require physical injury of the cell. Fluorescence based schemes can potentially facilitate sensing of intracellular oxygen using straightforward fluorometry or imaging schemes. However such platforms and probe chemistries are as yet largely underdeveloped.

A number of fluorescence and phosphorescence-based methodologies for sensing intracellular oxygen have been described, mainly using particulate sensors. Polymeric nanoparticles impregnated with oxygen-sensitive dyes (e.g. based on RuDPP or PtOEPK) loaded into cells by microprojectile delivery (Cao, Lee Koo et al. 2004; Koo, Cao et al. 2004); ‘lipobeads’ composed of a polymer particle (polystyrene) impregnated with a fluorescent dye (RuDPP) and a phospholipid shell incorporated into macrophages by phagocytosis (Ji, Rosenzweig et al. 2001); microspheres doped with PtTFPP introduced into large plant cells by microinjection (Schmalzlin, van Dongen et al. 2005); and the injection of hydrophilic metalloporphyrin dye complexed with albumin into skeletal muscle fibres (Hogan 1999) have been described. However, these systems are rather difficult to implement and they have serious limitations, including loading of the probe into the cell, sensitivity of the probe within the cell, even distribution of the probe throughout the cell, dye aggregation and compartmentation in the intracellular environment and/or high levels of phototoxicity due to high levels of singlet oxygen production.

To provide satisfactory performance, a probe for sensing intracellular oxygen should combine the following features: optimal photophysical properties and sensitivity to oxygen, simple, gentle and efficient means of delivery into the cell, minimal cyto- and phototoxicity and interference with cell function, minimal leakage from the cell and compartmentation. In addition, sensing of intracellular oxygen by fluorescence imaging with high spatial resolution and over prolonged periods of time requires a probe with high photostability. Furthermore, general convenience of use, flexibility and robustness of the probe and measurement procedure are the other important requirements.

The oxygen probes previously described above may possess some of these features but lack many other essential features. Particulate polymer-based probes have relatively large size, possess complex physical-chemical properties, and may have biocompatibility, stability and delivery issues. Their loading by projectile delivery, endocytosis or micro- or nano-injection is usually complex and inefficient and causes irreparable damage to the cell. Furthermore, random distribution of the relatively small number of particles within the cell may give a poor representation of the intracellular oxygen distribution. The use of molecular oxygen probes can potentially circumvent the limitations of particulate probes, particularly the problems of delivery into the cell, side effects on the cell and complexity of their synthesis and use. However, most of the soluble oxygen probes developed so far have limitations with respect to assessment of intracellular oxygen. The probes with relatively low molecular weight, such as free phosphorescent dyes, have substantial hydrophobicity, so that they partition within and/or leak from the cell. They can also bind to cell membranes, cause phototoxic and cytotoxic damage. Photostability of such probes is often insufficient for fluorescence microscopy applications and real-time live cell oxygen imaging with high spatial resolution. Delivery of such probes by microinjection is complex and damaging, so is cell loading with hydrophobic dyes. One of the problems associated with existing macromolecular oxygen probes is the problem of delivery of the probe to and/or into the cell.

The invention is directed towards providing an improved probe and methodology for sensing and imaging of intracellular oxygen.

### STATEMENTS OF INVENTION

According to the invention there is provided a probe for (in vitro) sensing and imaging of intracellular oxygen comprising an oxygen-sensitive fluorescent or phosphorescent dye linked to a macromolecular carrier; and a cell loading component or agent.

In one embodiment the macromolecular carrier and the cell loading component may be the same entity.

In another embodiment the dye may be covalently linked to the macromolecular carrier. For example, the dye may be conjugated to the macromolecular carrier.

In a further embodiment the dye, macromolecular carrier and cell loading component may be combined in one supramolecular structure.

The dye may be a highly photostable dye suitable for live-cell fluorescence microscopy measurements. The probe may comprise a phosphorescent oxygen-sensitive dye which is highly photostable under measurement conditions such as fluorescence microscopy, for example high-resolution live-cell fluorescence microscopy.

In one embodiment the oxygen-sensitive dye may be a phosphorescent platinum (II) porphyrin or palladium (II) porphyrin, a fluorescent complex of Ruthenium(II) or Osmium(II), or close analogs or derivatives of these dyes. The probe may be based on a Pt-coproporphyrin or a monofunctional reactive derivative thereof conjugated to a macromolecular carrier. The probe may be based on a monofunctional reactive derivative of Pt-coproporphyrin which facilitates conjugation to the macromolecular carrier.

The probe may be based on Pt(II)-coproporphyrin-ketone, a derivative or close analog thereof. Alternatively, the probe may be based on Pd(II)-coproporphyrin-ketone, a derivative or close analog thereof. The probe may be based on a stable Pt-chlorin or a stable Pd-chlorin.

The probe may contain two or more oxygen-sensitive dyes with different sensitivities to oxygen.

In one embodiment the macromolecular carrier may be a hydrophilic and biocompatible macromolecule. The macromolecular carrier may have a molecular weight in the region of 5,000-200,000 D. In one case the macromolecular carrier may be a polypeptide, a polynucleotide, a polysaccharide or a synthetic polymer such as poly(acrylate) or poly(ethyleneglycol). The polypeptide may comprise an inert protein such as serum albumin, for example bovine serum albumin (BSA), or an antibody or a fragment thereof. The polypeptide may be a cellular targeting polypeptide.

In one embodiment, the carrier may be specific to a cellular target, so that such probe has the ability to selectively accumulate in particular compartments within the cell, such as mitochondria, lysosomes, inner cell membrane(s), endoplasmic reticulum or at the cell surface.

The macromolecular carrier may have a net negative charge at physiological conditions.

In one embodiment, the cell loading agent may comprise a cationic liposomal or non-liposomal transfection agent. Alternatively, the cell loading moiety may comprise an agent which stimulates endocytosis and uptake of extracellular medium by the cells.

In a further embodiment the carrier and the loading entities can be one and the same. The supramolecular structure and/or the carrier and loading entity may be based on a polypeptide sequence, an antibody molecule or a part or fragment thereof. Such a probe may have the ability to recognise and bind to a specific target within the cell (an intracellular probe). Alternatively, the target can be on the surface of the cell (an extracellular probe).

The probe of the invention is designed to monitor and/or sense and/or image intracellular oxygen regardless of whether the probe itself enters a cell (intracellular probe) or remains outside a cell at the cell surface (extracellular probe).

As used herein, the term “loading” of cells with a probe equally applies to intracellular and extracellular probes. “Loading” in this sense is used to indicate that a probe is associated with a cell either intracellularly or extracellularly.

The probe may further comprise a biological buffer or medium which facilitates probe loading. In some cases the medium may contain special additives facilitating loading and cell survival. In other cases the medium may be protein-free.

The invention also provides the use of a probe of the invention for (in vitro) sensing of intracellular oxygen.

In another aspect the invention provides a method for (in vitro) sensing of intracellular oxygen comprising the steps of:
preparing a probe stock solution of desired concentration from a probe as described herein;
preparing a sample containing live cells in appropriate medium compatible with the probe;
adding probe solution to the cells and incubating to achieve cell loading;
analyzing fluorescence or phosphorescence from the cells loaded with the oxygen probe on a suitable detection system; and
assessing cellular oxygen on the basis of fluorescent or phosphorescent measurements.

In one embodiment loading of the cells with the probe may be performed at a concentration of oxygen-sensitive dye in the extracellular space in the region of 0.1-10 micromol/l. Alternatively, loading of the cells with the probe may be performed at a concentration of oxygen-sensitive dye in the extracellular space in the region of 10 nM/l to 100 μM/l.

The incubation time for loading the cells with the probe may be in the region of 0.2 to 24 hours such as 0.5-24 h.

In one case the cells may be loaded with the probe at a constant temperature of 30-37° C.

In one embodiment, after loading the cells and prior to fluorescence or phosphorescence measurement, the probe in the extracellular solution phase may be removed by washing the cells with medium.

In one case probe fluorescence/phosphorescence intensity signal from the loaded cells may be monitored and related to intracellular oxygen concentration. Fluorescent/phosphorescent signal from the loaded cells may be monitored in a time-resolved mode. Alternatively, fluorescence/phosphorescence lifetime of the probe may be monitored and related to intracellular oxygen concentration. Fluorescence/phosphorescence may be monitored kinetically over a period of time.

In one case fluorescent/phosphorescent measurements with the cells loaded with the probe may be performed in a microscopic volume. Fluorescence/phosphorescence measurements with the loaded cells may be performed on a fluorescent microscope. FLIM (fluorescence lifetime imaging microscopy) measurement mode may be used. The fluorescent microscope may be equipped with a pulsed laser or LED excitation and a fast gated CCD camera used in time-resolved mode. Confocal as well as multi-photon excitation fluorescent microscopy may be used.

In one embodiment individual cells are monitored. In another embodiment sets of individual cells may be monitored in one experiment and analyzed for differential response to stimuli.

Fluorescence/phosphorescence measurements with the loaded cells may be performed in a macroscopic volume. A fluorescent spectrometer or reader compatible with the probe and sample(s) being tested may be used to conduct the measurements. Multiple samples containing loaded cells may be analysed in parallel, for example in multi-well plates.

In one case the cells may be a suspension of adherent eukaryotic cells, or primary cells. Alternatively, the cells may be prokaryotic cells.

Cells may be pre-treated with effector compound(s) and the effect on cellular oxygen may be examined by measuring probe fluorescence/phosphorescence. In one embodiment the effector compound(s) may be added to cells suring optical measurements and alterations in probe signal caused by the effector(s) may be monitored and related to cellular oxygen or oxygen consumption.

Fluorescent/phosphorescent measurements may be localised to a specific cellular compartment to which the probe is delivered.

The probe of the invention provides efficient transfer, distribution and retention of the oxygen-sensitive material to and/or into live cells, thus facilitating measurements of intracellular oxygen.

The invention further provides a method of preparing a cellular probe comprising the steps of:
conjugating an oxygen-sensitive fluorescent or phosphorescent dye to a hydrophilic macromolecular carrier;
purifying the conjugate; and optionally
adding a cell loading agent to the conjugate.

### BRIEF DESCRIPTION OF THE DRAWINGS

The invention will be more clearly understood from the following description of some embodiments thereof, given by way of example only with reference to the accompanying drawings in which:

FIG. 1: Normalised absorption (a) and emission (b) spectra of the PtCP-BSA (1), PtCPK-BSA (2) and PdCPK-BSA (3) conjugates. (c) Emission of 1 μM of the PtCPK-BSA conjugate in PBS at different O₂ concentrations: 100% (A), 48.7% (B), 24.4% (C), 9.7% (D), 2.4% (E) and 0% (F) of air saturation. (d) Lifetime calibration and Stem-Volmer plot (τo/τ) for the PtCPK-BSA conjugate in PBS. (e) Emission of PdCPK-BSA conjugate at 100% of air saturation (A) and 0% O₂ (B). (f) Lifetime calibration and Stern-Volmer plot for the PdCPK-BSA conjugate in PBS;

FIG. 2: Images of live SH-SY5Y cells loaded with PtCPK-BSA by means of Endoporter® and imaged by time-lapse live cell microscopy. Images recorded prior to (a) and 60 min after (b) deoxygenation of the sample by incubation in 100% N₂;

FIG. 3: Phosphorescence intensity profiles of fixed (a) and live (b) HeLa cells loaded by means of Escort-III agent with PtCPK-BSA (1), PtCP-BSA (2) and Oxyphor G2 (3). Excitation at 395, 380 and 430 nm respectively;

FIG. 4: Images of SH-SY5Y cells loaded with PtCPK-BSA prior to (a) and 100 second after (b) stimulation with 20 nM ryanodine;

FIG. 5: Phosphorescence intensity profiles of: (a) A549 (2) and (b) HeLa (1) cells loaded by means of Escort-III agent with PtCPK-BSA (1) and PdCPK-BSA (2), and treated with 1 μM valinomycin; A549 cells (c) loaded by means of Escort-III agent with PtCPK-BSA and treated with 1 μM valinomycin (VM) and 2 μM antimycin A (AM-A); A549 (d) A549 cells loaded by means of Escort-III agent with PtCPK-BSA and treated with 500 μM 4-chloro-m-cresol without (CMC) and with (PC-CMC) pre-treatment with procaine, arrows indicate the time of compound addition. (e) A549 cells loaded by means of Escort-III agent with PtCPK-BSA treated with different concentrations of ryanodine (2 nM, 20 nM, 200 nM and 2 μM);

FIG. 6: Images of HeLa cells, loaded with PdCPK-BSA-Endoporter probe, prior to (a) and 800 sec after (b) treatment with 1 μM valinomycin; and

FIG. 7: Fluorescence lifetime analysis of Jurkat T-cells, loaded with PtCP-BSA-Escort probe and treated with 1 μM valinomycin (white bars) and 1 μM antimycin A (hatched bars). Control (black bars) represents the change in lifetime of extracellular PtCP following 1 μM valinomycin treatment.

### DETAILED DESCRIPTION OF THE INVENTION

The invention describes a probe and method for (in vitro) measurement of intracellular oxygen concentration and dynamic changes in cellular oxygen in biological samples containing live cells. In one embodiment the oxygen-sensitive probe is optimized for loading intracellularly (intracellular probe). In an alternative embodiment the oxygen-sensitive probe is optimised for loading extracellularly (extracellular probe). Both the intracellular and extracellular probes are configured to sense and monitor intracellular oxygen. The probe comprises a fluorescent or phosphorescent oxygen-sensitive dye conjugated to a hydrophilic macromolecular carrier such as a polypeptide or polysaccharide or synthetic polymer (e.g. polyethylene glycol); and an appropriate cell loading agent. The cell loading agent is an integral part of the probes of the invention. The cell loading agent may be linked to an oxygen sensitive dye-macromolecular carrier conjugate or it can be part of a supramolecular structure which contains the dye conjugated to the macromolecular carrier. Such a probe, when added to samples containing live cells or tissue, provides efficient, gentle and passive loading of the cells with the oxygen-sensitive material. In a different embodiment, the macromolecular carrier and cell loading agent are the same entity for example a peptide or the like such as an antibody or lectin. The joint macromolecular carrier and cell loading agent may be considered as a targeting entity for targeting the probe to a cell surface (extracellular probe). In this case the probe still retains the ability to ‘sense’ intracellular oxygen. Thus sensing of intracellular oxygen concentration can be realized combined with measurement of oxygen gradients within the cell and their dynamic changes linked to alterations of cellular metabolism caused by various stimuli.

The probe and measurement method of the invention are particularly suited to high-resolution live-cell fluorescent/phosphorescent imaging of oxygen distribution in individual cells. They can also be applied to populations of cells, i.e. macroscopic samples, with measurement on a fluorescent reader or spectrometer. The intracellular oxygen sensing probe and method can be used in areas such as general cell biology and physiology, pharmacology, medicine, biotechnology, drug discovery, biochemical and environmental toxicology.

The probes for sensing intracellular oxygen are based on conjugates of oxygen-sensitive photoluminescent or fluorescent dyes with macromolecular carriers. Some of the conjugates, for example those previously developed for extracellular use i.e. in bulk solution (Hynes, Floyd et al. 2003; O'Donovan, Hynes et al. 2005), were found to incorporate many features important for intracellular oxygen sensing. For example, they have hydrophilic nature and appropriate molecular size, which facilitate more simple and efficient loading procedures than those used with particulate probes. However these conjugates on their own lack a cell loading property. The conjugates of the present invention may comprise oxygen sensitive fluorescent or phosphorescent dyes with anionic hydrophilic macromolecules. Examples of anionic hydrophilic macromolecules include polypeptides, polynucleotides or biocompatible polymers all of which are among the preferred structures for this invention. Preferred examples include tetracarboxylic platinum(II)-coproporphyrin (PtCP) or it (mono)functionalised derivatives of these dyes (see e.g. U.S. Pat. No. 6,582,930), or palladium(II) and platinum(II)-coproporphyrin-ketones (PdCPK, PtCPK), or close analogs of these dyes, conjugated to polypeptide carriers such as serum albumin. Such conjugates have the following desirable qualities: highly soluble, inert, biocompatible, have acidic properties at physiological pH, and have good storage and operational stability. Other hydrophilic proteins and biomacromolecules, such as oligo- and poly-peptides, oligo- and poly-saccharides, oligo- and poly-nucleotides and synthetic polymers, such as poly(ethyleneglycol) and poly(acrylates), may also be used as carriers in such probes. Compared to the free oxygen-sensitive dyes, dye-carrier conjugates of the invention have a greatly reduced tendency to leak from or partition within the cells or damage the cells. Table 1 below sets out some examples of oxygen-sensitive materials used in the invention and their characteristics.

TABLE 1
Representative macromolecular conjugates (dye-carrier) used
in the probes of the invention, and their phosphorescent and
oxygen sensing characteristics in phosphate buffered saline (PBS).

| | λ_{exc} max. | λₑₘ max, | | |
|---|---|---|---|---|
| | (excitation | (emission | | |
| Conjugate | wavelength) | wavelength) | τ₀* | τₐ₋ₛ** |
| PtCP-Bovine | S = 380 nm | 646 nm | 81.3 μs | 22.6 μs |
| Serum Albumin | Q = 535 nm | | | |
| (BSA) | | | | |
| PtCP-Soybean | S = 380 nm | 646 nm | 80 μs | 21 μs |
| Trypsin | Q = 535 nm | | | |
| Inhibitor | | | | |
| PtCP-PEG, | S = 380 nm | 646 nm | 100 μs | 13 μs |
| Polyethylene | Q = 532 nm | | | |
| glycol, M.W. | | | | |
| 20,000 | | | | |
| PtCP-Wheat | S = 380 nm | 646 nm | 80 μs | 21 μs |
| Germ | Q = 532 nm | | | |
| Agglutinin | | | | |
| PtCP-Antibody | S = 380 nm | 646 nm | 81 μs | 24 μs |
| | Q = 532 nm | | | |
| PtCPK-BSA | S = 399 nm | 767 nm | 23.9 μs | 11.6 μs |
| | Q = 596 nm | | | |
| PdCPK-BSA | S = 405 nm | 796 nm | 138.8 μs | 19.6 μs |
| | Q = 607 nm | | | |

*Lifetime in deoxygenated solution;
**Lifetime in air-saturated solution;
S = Soret band and Q = Q band, these values are the maxima λ_{exc} values for the dyes in the conjugate.

In the invention, the hydrophilic oxygen-sensitive macromolecular conjugate(s) (dye and macromolecular carrier) is/are combined with an appropriate chemical or biological entity (cell loading agent), which provide efficient, gentle and relatively fast cell loading by passive means, to produce advanced fluorescence or phosphorescence based probes for sensing intracellular oxygen. In one embodiment the cell loading agent is selected to promote intracellular loading of the probe. For example, the cell loading entity can be a separate chemical or biological component, selected from the group of liposomal or non-liposomal cationic agents, or other compositions. Examples include the agents developed for transfection of eukaryotic cells with biomacromolecules such as nucleic acids and proteins, e.g. the Escort®, lipofectamine®, Fugene® families. Other examples of loading agents include those which stimulate endocytosis by the cells. These agents enhance the uptake of extracellular medium components and their accumulation in the cell. Thus far, such loading methods, reagents probe design and composition have not been used or described for intracellular oxygen sensing applications.

In an alternative embodiment the cell loading agent is selected to promote loading of the probe at the cell surface (extracellular probe). For example the cell loading agent may be an entity that specifically targets the probe to the cell surface such as a (poly)peptide configured to target the probe to glycoproteins at the cell surface or cell surface receptors or the like. Such probes of the invention are designed so that they incorporate, in one supramolecular structure, the key features required for both probe delivery to the cell and sensing of intracellular oxygen. For example, such probes can comprise an oxygen-sensitive dye conjugated to specific antibody, lectin or receptor molecules, which have the ability to selectively recognise certain cells and bind to their surface. These probes provide rapid and selective loading/staining of the cells, while their ability to sense oxygen locally, at cell surface can be used for the assessment of intracellular oxygen levels and their changes upon cell stimulation. Furthermore, such probes can be used to selectively stain and subsequently analyze intracellular oxygen gradients in particular population(s) of cells, without affecting other cell types in a mixture which would otherwise interfere oxygen measurements. For efficient loading of cells, concentrations of such probes in extracellular medium can be low (determined by affinity of the binding part, usually in the nM range), and the probe can concentrate at cell surface as a result of recognition and binding to the cell. We have shown, that these probes, when bound to the cells, retain the ability to ‘sense’ (intra)cellular oxygen. Some of such probes, namely the PtCP-Wheat germ agglutinin and PtCP-Antibody based probes, their properties and use for sensing cellular oxygen are given in Table 1 and in the Examples.

The correct selection of the fluorescent/phosphorescent dye and macromolecular carrier conjugate provides a probe with the desired physical-chemical and spectral properties, sensitivity to oxygen, electrical properties (molecular charge), high hydrophilicity and water-solubility, reduced photo- and cytotoxicity, retention and minimal leaching from the cells and even distribution within the cell. Whereas the cell loading chemistry and methodology provide simple, efficient, minimally invasive means of delivery of the oxygen sensing material to or inside live cells. These probes greatly facilitate preparatory and measurement procedures with the cells and allow new bioanalytical applications.

Such probe formulations are usable and provide optimal performance only within a certain range of concentrations/quantities of each component and under certain conditions of their use (medium, temperature, cell type, cell number, etc.). These parameters have been studied and optimised in the invention to provide advanced probes for sensing intracellular oxygen and physiological studies with different types of cells.

TABLE 2
Examples of probes of the invention and their use.

| | Loading | Probe formulation | | |
|---|---|---|---|---|
| Conjugate | agent | (conjugate/loading agent) | Cells | Medium |
| PtCPK-BSA | Escort | 1 μM/6 μl | A549 | Serum |
| | (Sigma) | | Jurkat | free |
| | | | HeLa | (S/F) |
| | | | | DMEM, |
| | | | | RPMI |
| | Endoporter | 1 μM/6 μM | A549, Jurkat, | DMEM, |
| | (Gene Tools) | | PC12, C2C12, | RPMI |
| | | | SH-SY5Y, | |
| | | | Human B cell Rat | |
| | | | Hepatocyte | |
| | | | HepG2, HeLa | |
| | Gene Juice | 1 μM/5 μl | A549 | DMEM |
| | (Merck) | | | |
| | Lipofectamine | 1 μM/5 μl | A549 | S/F |
| | (Invitrogen) | | | DMEM |
| PdCPK-BSA | Escort | 1 μM/6 μl | A549 | S/F |
| | | | Jurkat | DMEM, |
| | | | | RPMI |
| | Endoporter | 1 μM/6 μl | A549 | DMEM, |
| | | | Jurkat | RPMI |
| PtCP-BSA | Escort | 1 μM/6 μl | A549 | S/F |
| | (Sigma) | | Jurkat | DMEM, |
| | | | HeLa | RPMI |
| | Endoporter | 1 μM/6 μM | A549, Jurkat, | DMEM, |
| | (Gene Tools) | | PC12, C2C12, | RPMI |
| | | | HeLa, HepG2 | |
| | | | SH-SY5Y, | |
| | | | Human B cell, Rat | |
| | | | Hepatocyte | |
| PtCP-PEG- | Endoporter | 1 μM/6 μM | A549 | DMEM |
| 20k | (Gene Tools) | | | |
| PtCP- | Wheat germ | The carrier and | Jurkat, | RPMI, |
| wheat germ | agglutin | cell loading agent | Human B cell | other |
| agglutin | | are the same entity | | |
| PtCP- | monoclonal | The carrier and | Human B cell | RPMI, |
| monoclonal | antibody to | cell loading agent | | other |
| antibody to | CD19 cell | are the same entity | | |
| CD19 cell | surface marker | | | |
| surface | | | | |
| marker | | | | |

Formulation of the probe can be performed for example by mixing in a required proportion its components dissolved in a suitable solvent or medium, to achieve the desired concentrations for optimal loading of the cells. Alternatively, the probe can be formulated as a dry mixture of components which, when reconstituted in a medium, produces probe stock of desired composition and concentration. Some components can be provided in a solution form, while the others—in a dry form, so that when combined they produce the oxygen probe formulation for intracellular use. Some examples of the probes are given in Table 2.

When formulating and using probes of the invention, it is important to consider the issues of compatibility of the components of the probe, the medium used to produce probe formulation, the cell type and the growth medium used for culturing the cells. For example, some probes for example those which contain Escort III as loading agent, are not very compatible with high protein content media, which can interfere with loading process. In such cases the use of serum-free medium would be required.

Using the oxygen probes of the invention, loading of the cells with the oxygen-sensing material can be achieved in a minimally invasive passive manner. Normally probe formulation is added to a sample containing cells, medium and, if required, other components (e.g. effector compound, drug, additive, nutrient, etc.), and the sample is incubated over a period of time. Optimal time for loading the cells depends on the type of the probe, the cells, the medium and type of experiment. It is typically in a minutes to hours range. Incubation and cell loading are usually conducted such that cell viability and normal growth are preserved. When sufficient loading of cells is achieved, excess probe in the extracellular environment (probe that has not been loaded) can be removed by washing. Using such oxygen probes and procedures of the invention, efficient loading of live cells with the oxygen-sensing material is achieved, with minimal damage to the cells and minimal interference of cellular function. This makes it possible for subsequent analysis of loaded cells for intracellular oxygen and its dynamics, using appropriate measurement method and technique. Compared to the other oxygen-sensitive probes and loading techniques previously described, this invention allows more efficient, simple and reproducible loading of the cells, achieved at relatively low concentrations of the oxygen-sensitive material and with less stress to the cells. If the loading entity is not present in the probe, loading of the cells with the oxygen-sensitive material is usually drastically reduced.

As described above, the preferred probes of the invention are those based on the conjugates of hydrophilic (soluble) and biocomparible macromolecular carriers with suitable fluorescent/phosphorescent oxygen-sensitive dyes. It is known that the use of unconjugated (free) dyes for intracellular oxygen sensing instead of macromolecular conjugates is associated with pronounced side effects, such as aggregation of the dye, partitioning and accumulation in cell membranes and organelles, intrinsic cyto- and phototoxicity linked to the production of singlet oxygen (Papkovskii, Savitskii et al. 1990). To eliminate or at least to reduce these adverse effects, it is advantageous to covalently attach the oxygen-sensitive dye to a hydrophilic macromolecular carrier, such as polypeptide, polynucleotide, polysaccharide or synthetic polymer (poly(ethyleneglycol) or poly(acrylate)). For some probes of the invention, anionic macromolecular carriers such as bovine of human serum albumin (pI˜5), poly- and oligonucleotides (bear multiple phosphate groups), polyanionic polymers such as poly(acrylate), poly(glutamate) are advantageous, as they promote cell loading with high efficiency.

For use in the intracellular environment, the fluorescent/phosphorescent oxygen probes of the invention need to be able to effectively sense the oxygen within the cell and its dynamic changes within the physiological range of oxygen concentrations (0-100% or air-saturation or 0-250 μM) and conditions (medium, temperature). Therefore it is preferred that such fluorescent or phosphorescent macromolecular conjugates are moderately quenched by oxygen under corresponding experimental conditions. These conditions usually resemble physiological buffer and/or cell cytosol: neutral pH, high protein concentration, temperature 30-37° C. The optimal degree of quenching for such sensing materials, i.e. the change in fluorescence/phosphorescence intensity and lifetime when changing from deoxygenated to air-saturated solution, is considered to be between 1.5-fold and 10-fold. Oxygen probes having either too high or too low sensitivity to oxygen are less suited for sensing small changes in intracellular oxygen than the probes with optimal sensitivity specified above. However, for some systems and for experiments under hypoxia conditions (below 10% of air-saturation), probes with higher sensitivity to oxygen can be deployed. Furthermore, to achieve more uniform optical response within a broader range of oxygen concentrations, two conjugates with different sensitivity to oxygen (e.g. high and low) can be used in one intracellular oxygen probe formulation. Examples include a mixture of conjugates of Pt-coproporphyrin and Pd-coproporphyrin with serum albumin.

The selection of the oxygen-sensitive dye for the probe is critical. Platinum(II) and palladium(II)-porphyrins, their derivatives or close analogs are highly suited for this invention, particularly Pt-coproporphyrins (PtCP) which have convenient spectral characteristics and, upon conjugation to protein and polymeric carriers, display optimal sensitivity within the physiological oxygen range (0-21 kPa or 0-100% of air-saturation) (Hynes, Floyd et al. 2003). Oxygen probes based on PtCP-albumin or similar conjugates (see Table 1) can be used for sensing intracellular oxygen. Assessment of populations of cells can be performed on a fluorescent spectrometer or fluorescent plate reader, such applications are described in the Examples.

Oxygen-sensitive materials based on PtCP, as well as many other porphyrin dyes are problematic for high-resolution live cell fluorescent microscopy imaging, due to their insufficient photostability. To address this problem and enable intracellular oxygen imaging applications, dedicated oxygen probes have been developed as part of this invention, which are based on hydrophilic porphyrin-ketones, namely Pt- and Pd-coproporphyrin-ketones (PtCPK and PdCPK). As opposed to the hydrophobic porphyrin-ketones (e.g. Pt-octaethylporphine ketone) which have been used in solid-state oxygen sensors (Papkovsky and Ponomarev 1998; O'Riordan, Buckley et al. 2000; Koo, Cao et al. 2004), water-soluble porphyrin-ketones such as PtCPK and PdCPK have so far not been exploited or considered for conjugation to biomacromolecules and for use in sensing intracellular oxygen and imaging applications. In this invention we have demonstrated that PtCPK and PdCPK dyes and their conjugates with macromolecular carriers such as serum albumin are advantageous in many respects compared to other oxygen-sensitive materials previously described. Probes based on PtCPK or PdCPK dyes are particularly suited for sensing oxygen by live cell fluorescent micrcoscopy with high spatial resolution as they are superior to the other materials previously described.

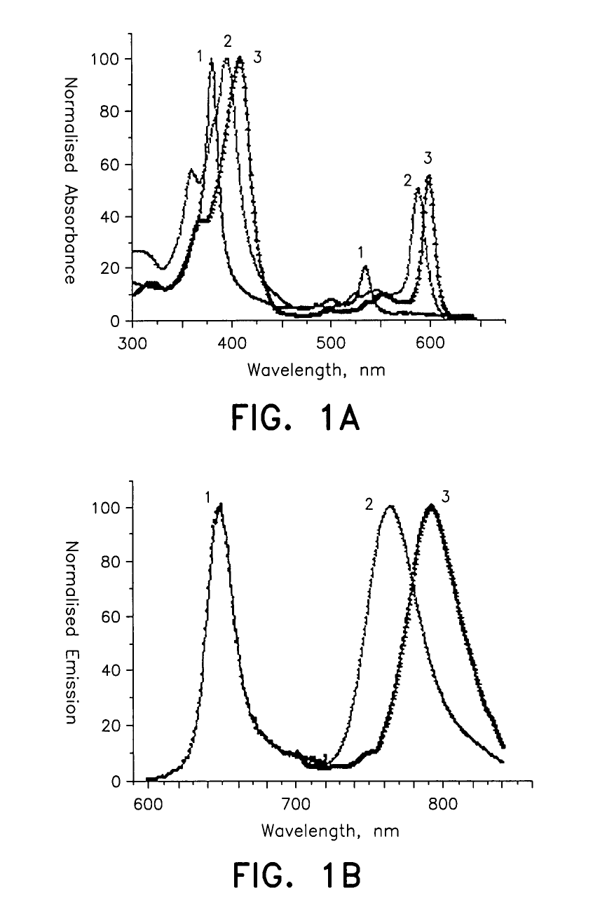

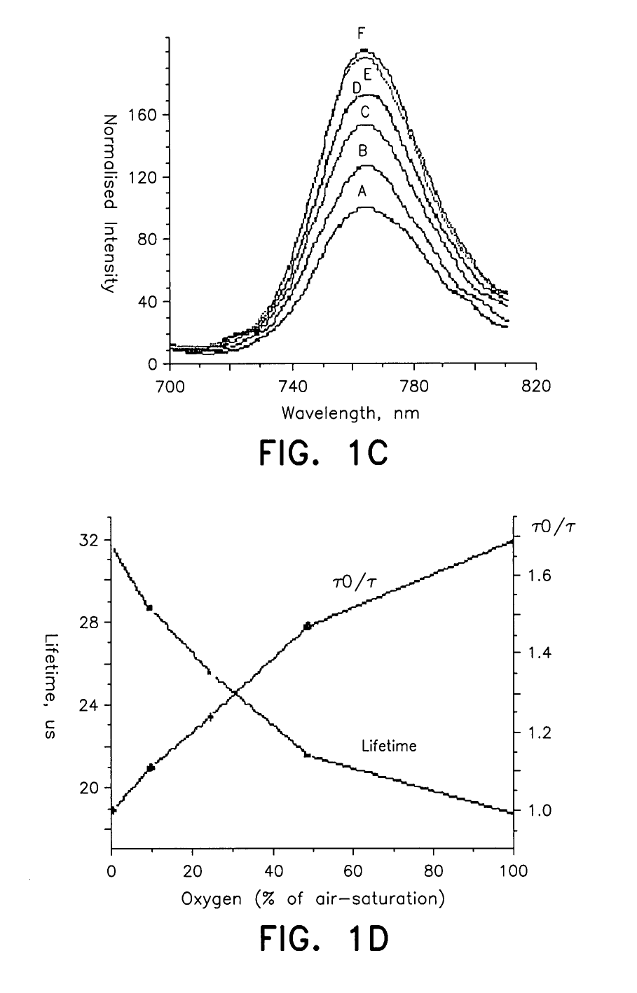

The photophysical properties and sensing characteristics of the PtCPK and PdCPK conjugates are shown in Table 1 and their absorption and emission spectra are shown in FIGS. 1a and 1b in comparison with the PtCP conjugates. The PtCPK and PdCPK conjugates and probes display intense absorbance bands and can be effectively excited at 370-410 nm (Soret band) or at 580-600 nm (Q-band). The probes emit (phosphoresce) in the near-infrared with maxima at 767 nm and 796 nm respectively. The quantum yields are smaller than for PtCP probe, while the phosphorescence lifetimes are 3-fold shorter for PtCPK and 2-fold longer for PdCPK. These probes are compatible with different types of imaging systems including wide field, confocal and/or, laser based, fluorescent microscopes, and more specialised time-resolved and FLIM detection configurations.

The sensitivity to oxygen for the PtCPK and PdCPK probes is within the optimal range and close to that of the PtCP probe (Hynes, Floyd et al. 2003). For the PtCPK probe the changes in phosphorescence intensity over the entire physiological oxygen range (0-100% of air-saturation or 0-21 kPa) is shown in FIG. 1c. This probe displays an overall change in intensity of 2.14 with a measurable change between 2.5% and 0% of air-saturation. Phosphorescence lifetime measurements showed a slightly less 1.7 fold quenching at 100% air-saturation. Calibration function (measured on a spectrometer) and Stern-Volmer plots are presented in FIG. 1d. The PdCPK probe displayed a greater 4.44-fold signal change between 100% and 0% of air saturation, its intensity and lifetime changes are shown in FIGS. 1e,f. The PdCPK probe is more suitable sensing and imaging intracellular oxygen over a low range.

Simple, highly efficient and reproducible loading of a number of live mammalian cells of different types, including suspension and adherent cell lines and primary cells with the oxygen probes of the invention has been demonstrated. Using BSA conjugates of PtCP, PtCPK and PdCPK formulated with liposomal transfection agents Escort™ (Sigma), Lipofectamine™ (Invitrogen) and Gene Juice (Merck) or fusion peptides such as Endoporter (Gene Tools), high, reproducible loading of A549, Jurkat, C2C12, Human B cells, primary hepatocytes, SH-SY5Y PC12, HeLa,cells was achieved. After such loading cell viability was not noticeably affected.

Probe loading was initially assessed by measurement on a fluorescent lifetime spectrometer of the PtCP probe, both in the extracellular medium and in the intracellular environment (i.e. after loading and washing of cells). A sample of Jurkat cells with extracellular PtCP probe displayed a lifetime of 24 μs, equivalent to air-saturated oxygen concentration. In loaded cells the probe lifetime was 31 μs indicating a reduced level of intracellular oxygenation. This illustrates both efficient and reproducible loading of the PtCP probe into the cells (phosphorescent signal corresponded to approximately 10 nM concentration), and shows that loaded cells were viable as they maintained oxygen gradients. No probe leakage from the loaded cells was detected. Similar behaviour was observed in cells loaded with the PtCPK and PdCPK based probes, which are of the same nature as the PtCP probe.

Samples of A549 and HeLa cells were loaded with PtCPK and PdCPK based probes and then analyzed by fluorescence microscopy. Example 3 illustrates that the intracellular probes and loading technique provide sufficiently high, even and reproducible loading of the cells, making it possible for a detailed examination of the cells by fluorescence microscopy and measurement of intracellular oxygen levels and their dynamic changes within the live cells.

The issue of probe photostability was also examined. The PtCPK and PdCPK based probes were tested in comparison to a PtCP based probe and a commercially available oxygen probe Oxyphor G2 (available from Oxygen Enterprises Limited, Philadelphia, USA). Live HeLa cells were loaded with different probes, then fixed and analysed on a fluorescent microscope. Over a 20-25 min time frame the PtCPK probe was seen to remain stable, whereas both PtCP and Oxyphor G2 probes degraded considerably. The photostability of the PtCPK probe was also conserved in live cells, while the instability of the PtCP and Oxyphor G2 probes was amplified, possibly due to the increased effect of singlet oxygen photogeneration. Intracellular imaging of the MeCPK probes using confocal fluorescence microscopy further verified their stability under the excitation with powerful 405 nm laser.

For some applications, it is advantageous that the oxygen-sensitive material, once delivered into the cell, distributes evenly in the cytosol, without leaking from the cell or partitioning. This allows probing of O₂ in different cells or parts of the cell by live cell fluorescent microscopy. As shown in the Examples below, probes of the invention based on macromolecular conjugates of the phosphorescent dyes, provide these features and ensure reliable measurement of oxygen levels and oxygen gradients in different types of cells and, using for example confocal fluorescent microscopy, in different compartments of the same cell.

Furthermore, the probe of the invention can be designed so that, once delivered into the cell, it has the ability to accumulate preferentially in particular sub-cellular compartments, such as mitochondria, endoplasmic reticulum, nucleus, etc. For example, the intracellular oxygen probe can be targeted to mitochondria by incorporating in the structure of the macromolecular conjugate a polypeptide sequence which is known to facilitate and/or be responsible for the delivery of endogenous proteins to mitochondria. Alternatively, the oxygen-sensitive dye can be directly conjugated to such a sequence or polypeptide. The probes of such kind are described in the Examples, they facilitate the sensing of local oxygen concentrations and sub-cellular oxygen gradients.

The methodology of intracellular oxygen sensing and imaging described in this invention has been successfully applied to monitoring of dynamic changes in intracellular oxygen in different types of mammalian cells. Adherent A549 and HeLa cells were grown under normal conditions, loaded with probes of the invention and subjected to various conditions or treated with various effector compounds with known mechanisms of action on cell metabolism while observing them under a fluorescent microscope. The probes were shown to be capable of detecting alterations in cell metabolism induced by various effectors. In such experiments, the probes themselves showed no significant cyto- and phototoxic action on the cells, viability of the loaded cells was similar to untreated ones. Additional details are given in the Examples.

The invention is further illustrated with non-limiting examples given below.

### EXAMPLES

### Example 1

### Fabrication of the PtCP Based Oxygen-Sensing Probe

PtCP-NCS dye was dissolved in DMSO to a concentration of 3 mg/ml (2.97 mM). 40 μl of this solution was added to 960 μl of bovine serum albumin in 0.05 M carbonate buffer, pH 9.6 and incubated for two hours at room temperature. The dye-BSA conjugate was separated from unbound dye on a PD10 desalting column in phosphate buffer saline. The conjugate fraction was collected and the concentration, and degree of labelling were determined from its absorption spectrum. The PtCP-BSA conjugate was dialyzed against water, lyophilized and stored dry at +4° C. for further use.

To produce the phosphorescent probe for intracellular oxygen sensing applications, the PtCP-BSA conjugate was dissolved in water at 100 μM solution. The intracellular oxygen probe was prepared by mixing 200 μl of serum free medium (RPMI) with 5 μl of Escort III transfection agent stock solution (Sigma) and 10 μl of the PtCP-BSA conjugate stock. For loading of mammalian cells, the probe solution was pre-warmed by incubating at 37° C. for 15 min and then used as described in Example 3. For further use, probe solution can be stored at +4° C. for several days.

Similarly, intracellular oxygen probes were prepared by formulating PtCP-BSA and PtCP-PEG conjugates with Endoporter loading agent (Gene Tools). 10 μl of 100 μM of the conjugate stock were added to 1 ml of serum containing medium and mixed with 6 μl of Endoporter.

### Example 2

### Fabrication of PtCPK and PdCPK Based Oxygen Probe

0.5 mg of either PtCPK or PdCPK free acid were dissolved in 0.2 ml of dimethylformamide, mixed with 1 mg of EDAC carbodiimide and incubated 15 min at room temperature to activate the carboxylic groups of the dye. Activated PtCPK or PdCPK was then added to a solution of BSA (1 ml, 10 mg/ml) in 0.1 M Na borate buffer, pH 8.5, agitated for two hours at room temperature followed by purification of the dye—BSA conjugate on a PD-10 desalting column as described in Example 1.

Chemical composition and concentration of the conjugate (dye:protein ratio) were determined spectrophotometrically.

To produce the probe for intracellular oxygen sensing and imaging applications, the PtCPK-BSA or PdCPK-BSA conjugate was reconstituted in water at 100 μM concentration. 200 μl of serum free medium were mixed with 5 μl of Escort III transfection agent and with 10 μl of the conjugate stock (final concentration of the conjugate—5 μM). For the loading of mammalian cells, the probe solution was pre-warmed for 15 min at 37° C. and then used with cells as described in Example 3.

Similarly, intracellular oxygen probes were prepared by formulating the PtCPK-BSA and PdCPK-BSA conjugates with Endoporter loading agent (Gene Tools). 100 μl of 10 μM of the dye-BSA conjugate stock were added to 1 ml of serum containing medium and mixed with 6 μl of Endoporter.

The intracellular oxygen probes were also prepared by formulating an equimolar mixture of the PtCPK-BSA and PdCPK-BSA conjugates with Fugene transfection agent. 45 μl of 90 μM PtCPK-BSA and 45 μl of 90 μM PdCPK-BSA were mixed with 6 μl of Fugene and made to a final volume of 200 μl. This probe stock was incubated for a minimum of 15 min at room temperature or 37° C. and then used with the cells as described in Example 3.

Probe formulations described in Examples 1 and 2 were used to load the cells and perform sensing experiments, as described in Examples 3 to 8.

### Example 3

### Loading of Live Mammalian Cells with PtCP-BSA Based Probe and Measurement of Intracellular Oxygen Concentration on a Fluorescent Spectrometer

A549 and HeLa cells were cultured in 75 cm² adherent cell flasks in DMEM supplemented with 10% FBS, 2 mM L-glutamine, 100 U/ml penicillin and 100 μg/ml streptomycin. 24 hours prior to loading, A549 cells were removed from the flask surface using PBS containing 2 mM EDTA and 1× trypsin, and aliquotted in 1 ml volumes into 35 mm glass bottom dishes (Mattek) or 10 mm round glass coverslips (Scientific Laboratory Supplies). Jurkat T-cells were grown in RPMI medium supplemented with 10% FBS, 2 mM L-glutamine, 100 U/ml penicillin and 100 μg/ml streptomycin.

For loading, the PtCP-BSA based probe comprising PtCP-BSA conjugate formulated with Escort III (described in Example 1) was used. Probe solution was pre-warmed by incubating at 37° C for 15 min followed by addition of 100 μl of the probe solution to the cell culture dish containing 800 μl of serum free medium. The cells and probe solution were incubated for 5 hours in a 5% CO₂ incubator at 37° C., cells were then washed with 1 ml of serum free medium three times.

Loading of the cells with an intracellular probe comprising PtCP-BSA conjugate and Endoporter agent (similar to the one described in Example 1) was carried out as follows. HeLa cells cultured in 96-well plates in RPMI medium supplemented with 10% serum were washed with fresh medium followed by addition of probe formulation. The cells were incubated with PtCP-BSA-Endoporter probe for 18-24 hours followed by washing.

Loading of the cells loaded with the PtCP-based probe(s) was initially assessed by measurement on a fluorescent lifetime spectrometer Cary Eclipse (Varian), both in the intracellular environment (i.e. after loading and washing of the cells) and in the extracellular medium (at the start of loading, no washing). A sample of unloaded Jurkat cells with extracellular PtCP probe displayed a lifetime of 24 μs, equivalent to air-saturated oxygen concentration. In loaded cells under resting condition the probe lifetime was 31 μs. Upon addition of the mitochondrial inhibitor antimycin A, which is known to block oxygen respiration, probe lifetime was seen to decrease and level off at ˜25 μs. The results show a lower average level of oxygenation in the intracellular space (probe lifetime is inversely proportional to the local concentration of oxygen) before the addition of inhibitor. The inhibitor blocks oxygen consumption and adjusts intracellular oxygen levels to air-saturated levels. The addition of uncouplers such as FCCP or valinomycin (both at 1 μM concentrations) caused an increase in the probe lifetime from a basal level of 31 μs to 40-45 μs due to uncoupling of oxidative phosphorylation resulting in increased oxygen uptake and a resulting drop in intracellular oxygen concentration. These experiments illustrate efficient loading of the PtCP-BSA probe into the cells, where it produced phosphorescent signal which corresponded to ˜5 nM concentration, maintained oxygen gradients in viable cells and no probe leakage from the cells.

In a similar manner, loading of the other cell lines, including A549, C2C12 and cells, primary hepatocytes and cultured preimplantation mouse embryos, was carried out using the probes described in Example 1.

### Example 4

### Loading of Mammalian Cells with PtCPK and PdCPK Based Probes and Imaging of Intracellular Oxygen on a Fluorescent Microscope

Loading of the cells with the PtCPK-BSA and PdCPK-BSA based probes (as described in Example 2) was carried out in the same way as described in Example 3 for the PtCP-BSA probes (i.e. 5 h in serum-free medium for the Escort or 24 h in serum-rich medium for Endoporter). A549 and HeLa cells loaded with PtCPK-BSA and PdCPK-BSA probes were then washed and analyzed by fluorescence microscopy. Imaging experiments of cells loaded with the probes were carried out on an Olympus IX51 inverted fluorescence microscope equipped with 75 W Xenon Arc Lamp (Cairn), an Optoscan Monochromator (Cairn) and an Orca-ER CCD camera (Hamamatsu). Live cell imaging was carried out using an Olympus UplanFl 1.3 NA 100× oil-immersion objective. The images were analysed using Kinetic Imaging AQM Advanced software (Version 6). The PtCPK and PdCPK probes were imaged under excitation at wavelengths of 395 nm and 400 nm respectively collecting the phosphorescence emission using a 600 nm cut-off filter.

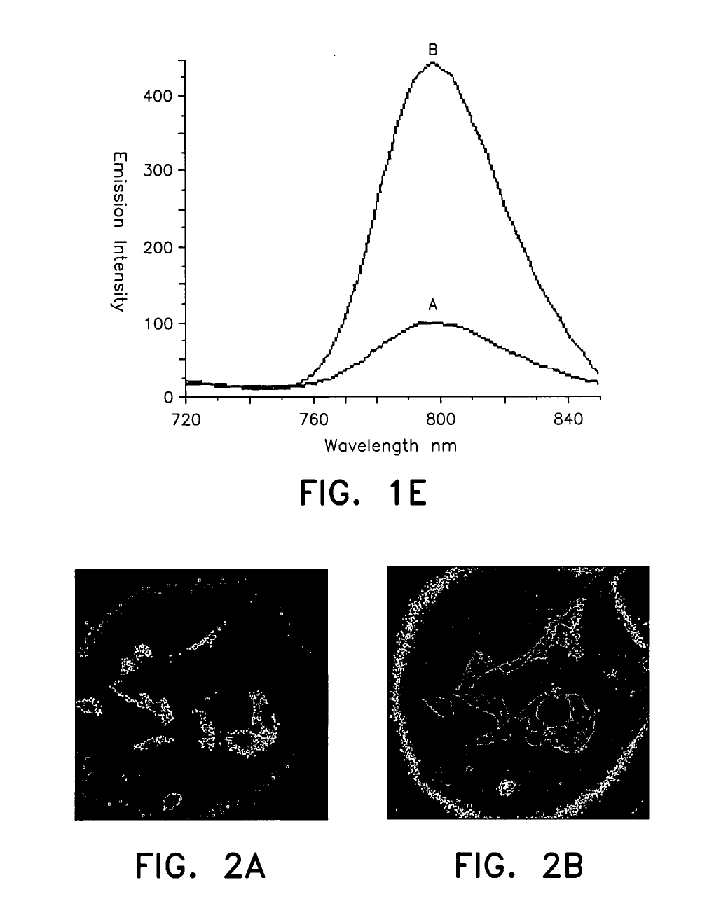

Imaging of loaded SH-SY5Y cells under conditions of normoxia (21% O₂) and anoxia (0% O₂) were carried out on a Zeiss Axiovert 200 equipped with a complete cell cultivation system (Pecon). The sample was subjected to 100% N₂ for one hour during which time-lapse imaging was carried out with images recorded every 60 sec. The probe was excited at 595 nm with emission collected using a 700 nm longpass filter. Images were recorded and analysed using ImSpector software (LaVision Biotech). FIG. 2 shows images recorded at time 0 minutes (t₀) and time 60 minutes (t₆₀).

### Example 5

### Assessment of Probe Photostability in Imaging Experiments

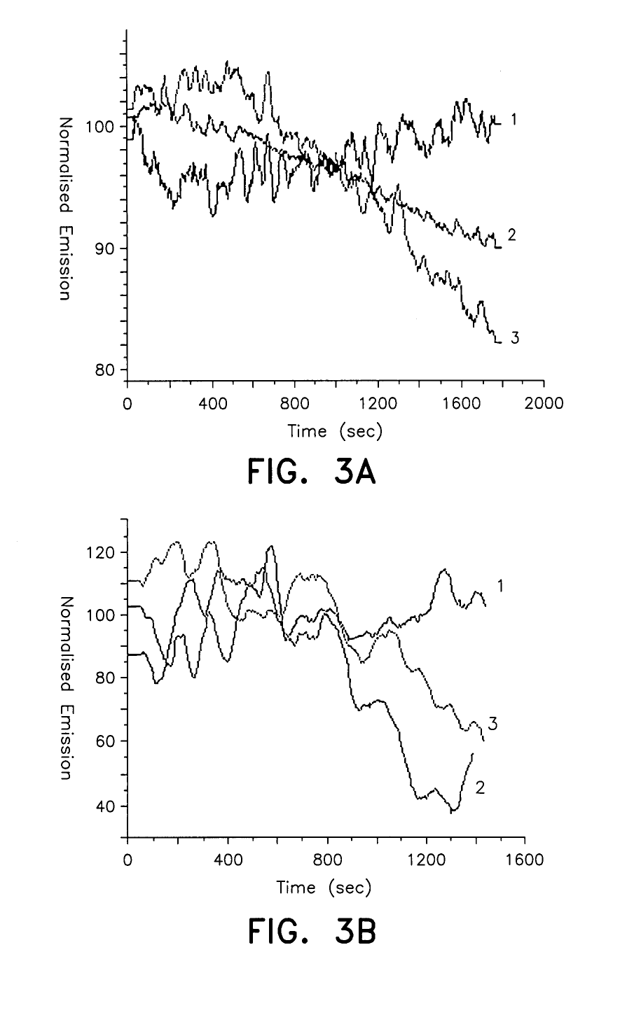

Photostability of the different porphyrin-based oxygen probes in conditions of typical live cell imaging experiments was examined comparatively. Live HeLa cells were loaded with probes (PtCP-BSA (as described in Example 1), PtCPK-BSA and PdCPK-BSA (as described in Example 2) conjugates formulated with Escort III), as described above in Examples 3 and 4. Cells were fixed and analysed on a fluorescent microscope. Oxyphor G2 probe (Dunphy, Vinogradov et al. 2002) was also included in the study. FIG. 3a shows that under continuous illumination at absorption maximum of each probe over a 20-25 min time frame, the PtCPK-BSA probe is substantially more stable than both the PtCP probe and Oxyphor G2. The photostability of the PtCPK probe was also conserved in live cells (see FIG. 3b) over 20 min of continuous excitation, while the instability of the PtCP-BSA probe and Oxyphor G2 was amplified, possibly due to the increased effects of reactive oxygen species photogenerated by the probes. Probes based on PtCPK and PdCPK dyes are more advantageous for microscopy imaging applications than those based on other oxygen-sensitive dyes as PtCPK and PdCPK have a greater photostability. The photostability of the PtCPK and PdCKP based intracellular probes was also demonstrated using confocal fluorescence microscopy with laser excitation at 405 nm.

### Example 6

### Loading of Mammalian Cells with PtCPK and PdCPK Based Probes and Monitoring Cellular Responses by Intracellular Oxygen Imaging

Adherent A549, HeLa and SH-SY5Y cells were grown under normal conditions. Cells were loaded with probes based on PtCPK-BSA and PdCPK-BSA and Escort III (described in Example 2), and treated with various effector compounds with known mechanisms of action on cell metabolism and cellular response to the effector compounds was observed under a fluorescent microscope.

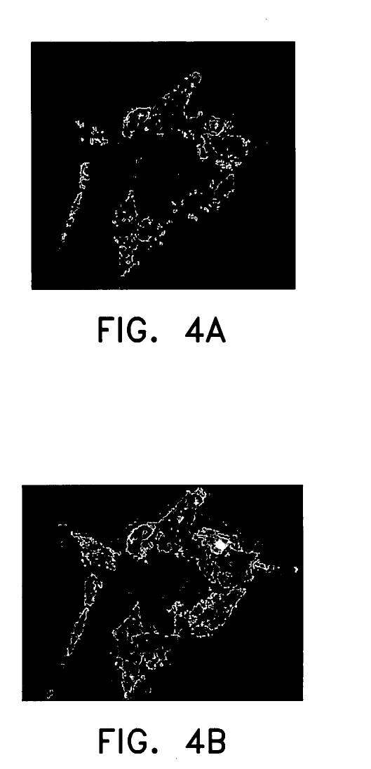

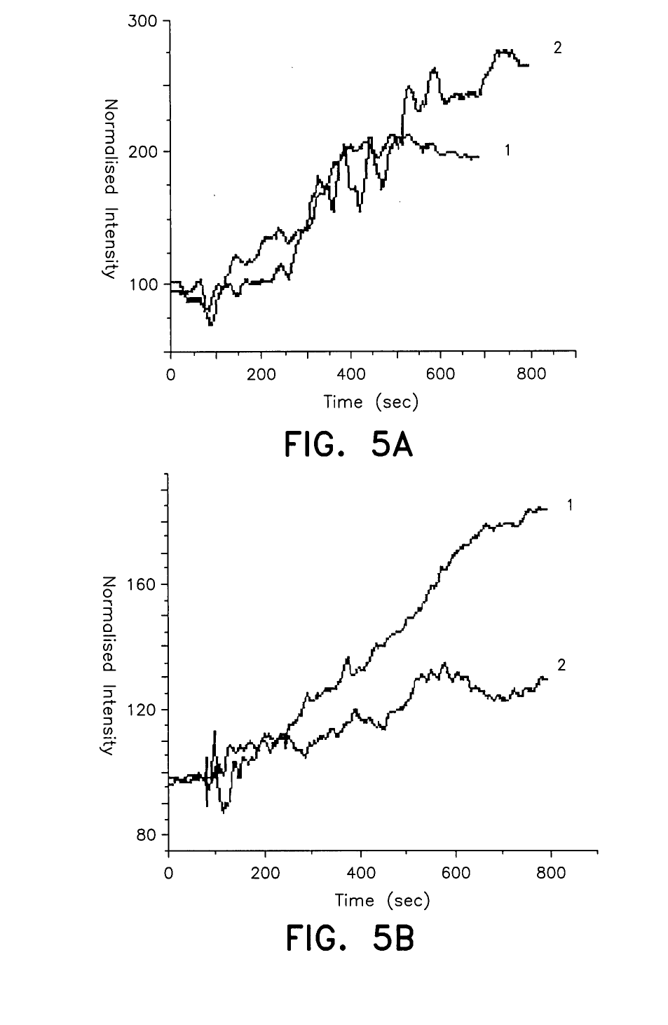

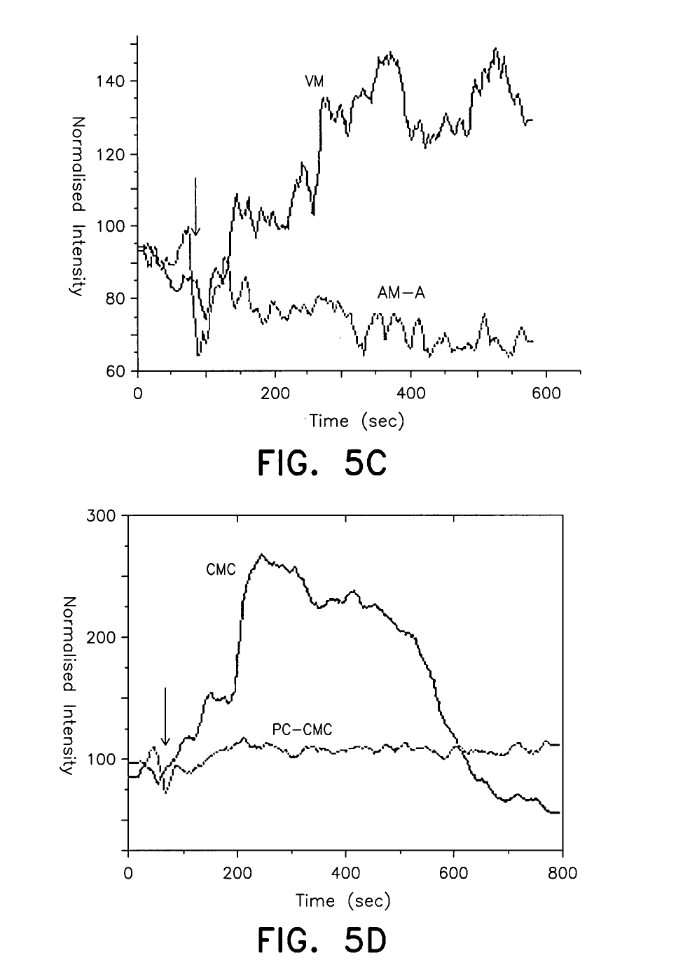

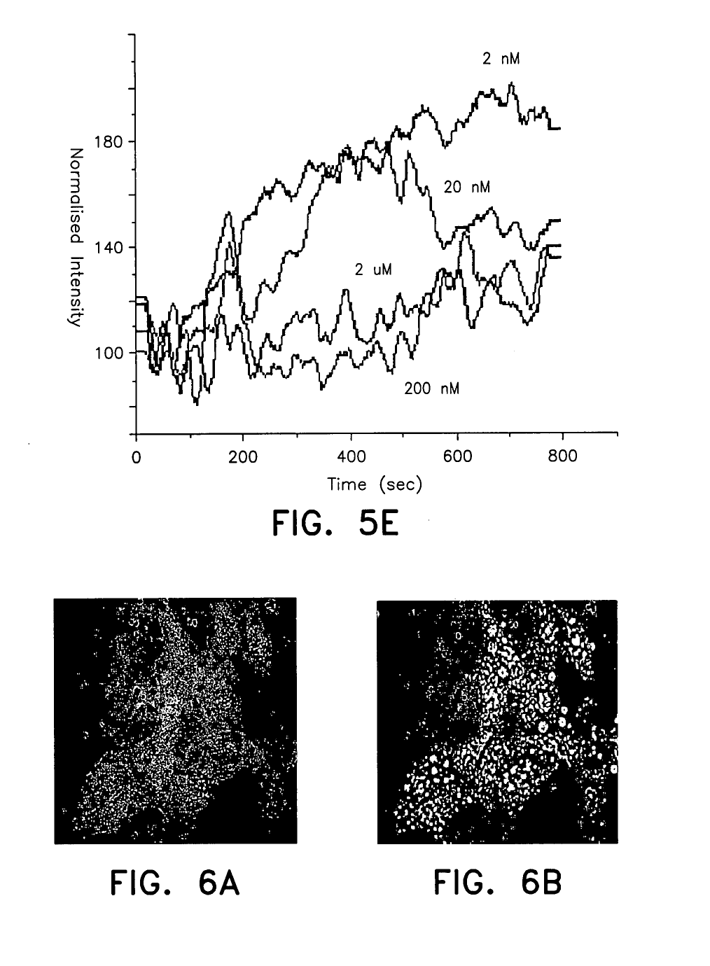

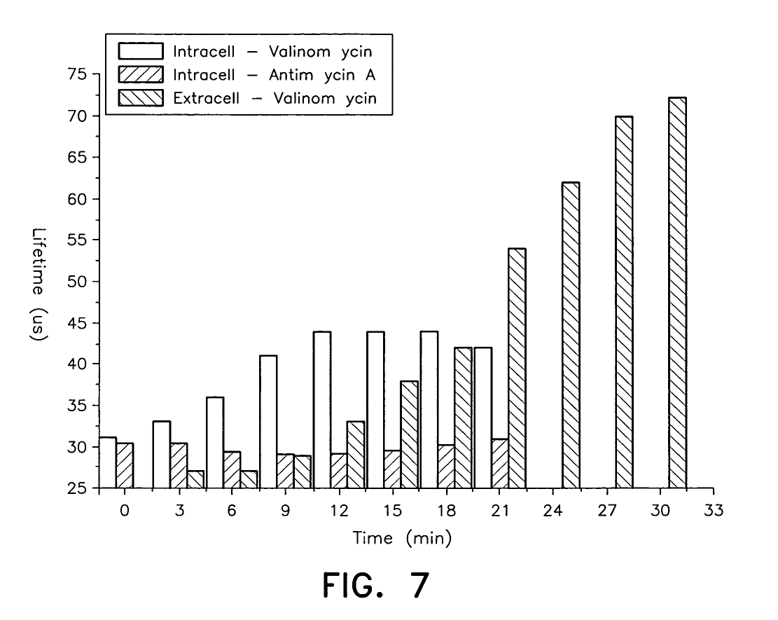

The effect of Ca-channel agonist ryanodine (20 nM) on SH-SY5Y cells, with up-regulation of cell metabolism by inducing a release of Ca2+ from intracellular stores, is shown in FIG. 4. The action of valinomycin on A549 cells and HeLa cells shown in FIGS. 5a and 5b illustrates the response to be rapid and cell specific. In the A549 cells the PtCPK and PdCPK probes displayed a similar response to valinomycin with a greater range displayed by the PtCPK probe. This indicates that the average base concentration of O₂ in respiring A549 cells was greater than 50% of air saturation. In the HeLa cells, greater sensitivity to the effects of valinomycin was displayed by the PdCPK probe (see FIG. 5b), indicating a lower base level of intracellular oxygen than in A549 cells. The effect of addition of 2 μM antimycin A on A549 cells is shown in FIG. 5c. An approximately 40% drop in PtCPK phosphorescence was observed as the base rate of respiration was inhibited resulting in higher levels of intracellular oxygen emanating from O₂ influx from the extracellular medium. Control experiments without valinomycin (pure DMSO) showed no change in PtCPK phosphorescence.

The A549 cell line possess type I ryanodine receptors (RYR 1) (Xue, Zhao et al. 2000), while recent studies have demonstrated specific use of 4-chloro-meta-cresol (CMC) for stored calcium release in this cell line (Padar, van Breemen et al. 2004). A pronounced effect on the phosphorescence following the addition of 500 μM CMC to a population of respiring A549 cells is seen in FIG. 5d. The specificity of the CMC response was confirmed by incubating A549 cells with an equimolar concentration of the ryanodine channel blocker procaine followed by CMC treatment. In this case, phosphorescence intensity increased by less than 10% after the introduction of CMC. FIG. 5e shows the response of A549 cells, loaded with PtCPK, to calcium release caused by different concentrations of ryanodine. Lower concentrations (2 nM and 20 nM) cause calcium release from intracellular stores through RYR 1 resulting in increased uptake of intracellular oxygen and increased phosphorescence. Higher concentrations (200 nM and 2 μM) are known to have an inhibitory rather than stimulatory effect on RYR 1 and thus little oxygen uptake is observed.

### Example 7

### Loading of Mammalian Cells with the PtCP Based Probe and Monitoring Cellular Responses by Phosphorescence Lifetime Sensing

Jurkat T-cell lymphoma cells were grown under normal conditions and loaded with the PtCP-BSA-Escort III based probe (as described in Example 1) using the method described in Example 3. Following washing the basal level of intracellular oxygen was assessed by measuring the phosphorescence lifetime of the PtCP probe using a Cary Eclipse spectrofluorometer and analyzing phosphorescence decays using Origin™ software (single-exponential fits). Phosphorescence lifetimes of the intracellular probe in loaded cells were measured at three minute intervals following the addition of 1 μM valinomycin. The increase in probe lifetime was significantly faster than in a control sample where no loading agent was used and the probe remained in the extracellular medium. A second sample of cells loaded with PtCP was treated with antimycin A, where a slight drop in intracellular probe phosphorescence lifetime was observed.

### Example 8

### Assessment of Intracellular Oxygen by Fluorescence Lifetime Imaging Microscopy (FLIM)

SH-SY5Y cells were loaded with the PtCPK-BSA-Endoporter probe (as described in Example 2) using the method described in example 3. Phosphorescence intensity and lifetime of the loaded cells was assessed using an Axiovert 200 fluorescence microscope (Zeiss) equipped with a LaVision Imager Pro lifetime imaging system including an intensified CCD camera with excitation using modulated 395 nm and 595 nm LEDS. Collection of emitted light was using a 700 nm longpass filter and image acquisition at a number of time points along the emission decay (between 10 μs and 90 μs) was achieved using ImSpector software (LaVision). Phosphorescence lifetime in individual resting SH-SY5Y cells was between 8.5 and 10 μs (an average of 10 cells). Following the introduction of anoxic conditions by purging the system with nitrogen for 60 min using a cell cultivation system (PeCon), the lifetime of intracellular PtCPK probe increased to 16.5-19 μs for the same cells.

### Example 9

### Preparation of the Probe Targeted to Cell Surface Molecules and Measurement Cellular Oxygen

Wheat germ agglutinin (WGA) at 1 mg/ml concentration in 0.1 M Na carbonate, pH 8.6 was labelled with the PtCP-NCS dye using a 20-molar excess of dye. After two hours of incubation at room temperature, the resulting conjugate was separated from the free dye as described in example 1 above. Main characteristics of the supramolecular PtCP-WGA probe are given in Table 1. Jurkat T cells and human B cells were incubated with WGA-PtCP probe at 1 μM concentration for 30 min at 37° C. in RPMI medium, to achieve binding of the probe to glycoproteins on cell surface. After washing of loaded cells with medium, local oxygen concentration (at cell surface and within the cell) was assessed. and responses to effectors of respiration such as 1 μM FCCP were measured. Lifetimes of the cells loaded with WGA-PtCP probe was measured on a time resolved fluorescent reader Victor², using excitation at 340 nm excitation and 642 nm emission filters, and pairwise intensity readings at delay times 30 and 70 μs (at gate time 100 μs). Basal lifetime of resting Jurkat cells was recorded as 25 μs, moving to 32 μs upon the addition of FCCP (uncoupler of respiration). Under the same conditions, using the PtCP-BSA conjugate which had no ability to bind to the cells and remained in extracellular medium, intracellular oxygen gradients and responses to effectors were undetectable.

Similarly, a conjugate of monoclonal antibody to the CD19 cell surface marker with PtCP dye was synthesized and purified (see characteristics in Table 1). The conjugate was used at a concentration of 100 nM in extracellular medium, to stain cultured B cells (known to have CD19 receptors), for 60 min at 37° C. After washing, probe lifetime was monitored on the Victor² fluorometer and local oxygen gradients for the resting and stimulated B cells were assessed. The probe was seen to produce changes in phosphorescence lifetime in response to cell treatment with different metabolic effectors such as FCCP. These changes reflected changes in intracellular oxygen due to enhanced or inhibited respiration of the cells.

### Example 10

### Preparation and Use of the Intracellular Oxygen Probes Targeted to Mitochondria

The PtCP-BSA conjugate, synthesized as described in Example 1 was additionally labelled with a synthetic oligopeptide containing the following sequence: VLTPLLLRGLTGSARRLPVPRAKAC. The peptide comprises a mitochondria targeting sequence (according to http://psort.ims.u-tokyo.ac.jp), a terminal cysteine group was used for the conjugation. Labelling with the peptide was achieved by means of a heterobifunctional reagent—succinimide ester of maleimidopropionic acid. The PtCP-BSA conjugate was incubated with 10-molar excess of the reagent in 0.1 M phosphate buffer, pH 7.8 for 4 hours at room temperature, then separated on a PD-10 column in the same buffer. The peak of activated PtCP-BSA was collected, mixed with 10-molar excess of the peptide, incubated for further 6 hours. Finally, the PtCP-BSA-peptide conjugate was separated on a PD-10 column.

Alternatively, the peptide was conjugated directly to PtCP. The monofunctional maleimide derivative of PtCP was used, which reacts spontaneously with —SH groups on the peptide (terminal cysteins) to form stable covalent linkage. The conjugation was achieved by incubating the peptide with 3-molar excess of PtCP-maleimide in DMSO, followed by HPLC purification on a reverse phase C18 column using a gradient of acetonitrile in TEAA buffer, pH6.5. The PtCP-peptide conjugate peak (identified by spectral analysis on the diode-array HPLC detector) was collected, pooled and dried by vacuum centrifugation. It was then used in intracellular oxygen sensing experiments.

Probes incorporating the PtCP-BSA-peptide and PtCP-peptide conjugates (successful labelling was confirmed by MALDI mass spectrometry) were formulated with Endoporter agent and used for cell loading as described in Examples 3 to 8. PC12 cells loaded with these probes produced high signals on the Victor reader. At the same time, for the resting loaded cells phosphorescence lifetimes were higher than those obtained with the PtCP-BSA probe, suggesting their different localization within the cell.

### Example 11

### Synthesis and Use of the Fluorescent Intracellular Oxygen Probes

Mono-amino poly(ethylene glycol), MW 20,000 (PEG, Shearwater Corporation) was dissolved at a concentration of 5 mg/ml in 3 ml of 0.1 M carbonate buffer, pH 9.5. To this solution 5-molar excess of bis-(2,2′-bipyridine)-(5-isothiocyanatophenantroline)ruthenium bis-(hexafluorophosphate) dye (Ru-NCS, Fluka) was added and the mixture was incubated for 6 hours at room temperature. Purification of the dye-PEG conjugate was carried out on a PD-10 desalting column in phosphate buffer saline. Conjugate peak was collected, desalted, dried and stored in the dark at 4° C.

The oxygen probe was prepared using a 10 μM stock solution of the Ru-dye-PEG conjugate in water, 100 μl of which were added to 1 ml of serum-containing RPMI medium, followed by the addition of 6 μl of Endoporter loading agent (Gene Tools). The resulting probe was used to load PC12 cells grown in mini-dishes, as described previously in Examples 1-3. After loading and washing, the cells were analysed on a fluorescence microscope, under 470 nm excitation and 620 nm emission filters. Addition of glucose/oxidase-glucose to the extracellular medium was seen to cause a measurable increase in fluorescence of loaded cells (due to sample deoxygenation), although signal drift was significant.

Overall, the invention provides the new family of intracellular oxygen probes and methodology for sensing cellular oxygen. It also demonstrates the efficiency of such probes and techniques and their use with different cell types and in different applications. They are particularly useful for the studies of mitochondrial and cellular function, physiological and metabolic responses of live mammalian cells. In such experiments, they demonstrate low photo- and cytotoxicity and no damage to the cells.

The PtCP based probes are well suited for simple, high throughput studies of cell populations, to study metabolic and physiological responses of different types of cells. Analysis can be performed on standard time-resolved fluorescent plate readers. These probes can also be used in conjunction with fluorescent imaging systems, in experiments which do not require very high photostability of the probe.

The probes based on PtCPK and PdCPK dyes are advantageous, particularly for high-resolution live cell oxygen imaging systems and experiments. The hydrophilic nature and peripheral functional groups enable conjugation of the dyes to macromolecular carriers, such as albumin and other biomacromolecules, to produce highly photostable and biocompatible macromolecular oxygen-sensitive materials. Corresponding near-infrared oxygen probes display optimal sensitivity to oxygen and compatibility with existing imaging equipment. They can be effectively excited with continuous wave or pulsed LEDs and lasers (e.g. 590 nm, 405 nm or 390 nm) and detected by CCD cameras. The hydrophilic and anionic nature of the conjugates and relatively small size facilitate simple loading into cells by passive transfection. Macromolecular carrier reduces partitioning of the oxygen-sensitive material in the intracellular environment, leaching, cyto- and phototoxic effects. These probes can be used in conjunction with standard fluorescence imaging systems, including basic wide field, confocal fluorescent microscopes, as well as on more specialised phosphorescence lifetime-based imaging systems (FLIM), to allow simple imaging analysis and real-time monitoring of intracellular oxygen in individual mammalian cells, both under resting conditions and upon stimulation.

The measurement of intracellular oxygen has a number of inherent advantages over techniques which measure extracellular oxygen gradients. High-resolution fluorescent imaging facilitates measuring changes in oxygen within individual cells, while the microplate based systems are only applicable to large populations of cells (10³ cells/well or greater in a sealed compartment) with end-point parameter readout. Furthermore, the kinetics of local changes in intracellular oxygen is much faster than formation of global oxygen gradients in bulk sample, which eliminates the need for exclusion of ambient oxygen and sealing test samples. Thus, subtle and transient metabolic responses, such as those linked to the intracellular calcium elevations, may be clearly seen by measuring intracellular oxygen, but are outside the sensitivity of extracellular sensing methodologies. Effector treatment of cells is also simplified in this case.

The invention provides a key technique with wide ranging applicability which can complement other important fluorescent probes for intracellular parameters such as calcium indicators, fluorescent protein tags, probes for reactive oxygen species and mitochondrial membrane potential, etc. It is amenable to high content screening applications. The long-decay emission of PtCPK and PdCPK based probes allows time-resolved fluorescent imaging in the microsecond time domain, which can further increase the sensitivity and contrast of the sensing system and facilitate multiplexing with other probes. Phosphorescence lifetime-based oxygen imaging can also be applied allowing more straightforward determination of the absolute oxygen concentrations and eliminating the need of frequent calibrations. These advanced techniques, which are known to specialists in this and related areas and which are relatively easy to implement, can further augment what is already an extremely useful tool for cell biologists to study intracellular oxygen and by extension mitochondrial and cellular function and dysfunction.

The invention is not limited to the embodiments described but may be varied in construction and detail.

### REFERENCES

Bacon, J. R. and J. N. Demas (1991). Apparatus for oxygen determination. U.S. Pat. No. 5,030,420.
Cao, Y., Y. E. Lee Koo, et al. (2004). “Poly(decyl methacrylate)-based fluorescent PEBBLE swarm nanosensors for measuring dissolved oxygen in biosamples.” Analyst 129(8): 745-50.
Dunphy, I., S. A. Vinogradov, et al. (2002). “Oxyphor R2 and G2: phosphors for measuring oxygen by oxygen-dependent quenching of phosphorescence.” Anal Biochem 310(2): 191-8.
Eden, G. and N. M. Sullivan (1992). Detecting microbiological growth. U.S. Pat. No. 5,232,839.
Hogan, M. C. (1999). “Phosphorescence quenching method for measurement of intracellular PO2 in isolated skeletal muscle fibers.” J Appl Physiol 86(2): 720-4.
Hynes, J., S. Floyd, et al. (2003). “Fluorescence-based cell viability screening assays using water-soluble oxygen probes.” J Biomol Screen 8(3): 264-72.
Hynes, J., R. Hill, et al. (2005). “The use of a fluorescence-based oxygen uptake assay in the analysis of cytotoxicity.” Toxicol In Vitro.
Hynes, J., L. D. Marroquin, et al. (2006). “Investigation of Drug-induced Mitochondrial Toxicity using Fluorescence-based Oxygen-sensitive Probes.” Toxicol Sci.
Ji, J., N. Rosenzweig, et al. (2001). “Molecular oxygen-sensitive fluorescent lipobeads for intracellular oxygen measurements in murine macrophages.” Anal Chem 73(15): 3521-7.
John, G. T., I. Klimant, et al. (2003). “Integrated optical sensing of dissolved oxygen in microtiter plates: a novel tool for microbial cultivation.” Biotechnol Bioeng 81(7): 829-36.
Khalil, G. E., M. Gouterman, et al. (1989). Method for measuring oxygen concentration. U.S. Pat. No. 4,810,655.
Koo, Y. E., Y. Cao, et al. (2004). “Real-time measurements of dissolved oxygen inside live cells by organically modified silicate fluorescent nanosensors.” Anal Chem 76(9): 2498-505.
Lubbers, D. W. and N. Optiz (1977). Method and arrangement for measuring the concentration of gases. U.S. Pat. No. 4,003,707.
O'Donovan, C., J. Hynes, et al. (2005). “Phosphorescent oxygen-sensitive materials for biological applications.” Journal of Materials Chemistry 15(27-28): 2946-2951.
O'Mahony, F. C., C. O'Donovan, et al. (2005). “Optical oxygen micro-respirometry as a platform for environmental toxicology and animal model studies.” Environmental Science & Technology 39: 5010-5014.
O'Riordan, T. C., D. Buckley, et al. (2000). “A cell viability assay based on monitoring respiration by optical oxygen sensing.” Anal Biochem 278(2): 221-7.
Padar, S., C. van Breemen, et al. (2004). “Differential regulation of calcium homeostasis in adenocarcinoma cell line A549 and its Taxol-resistant subclone.” Br J Pharmacol 142(2): 305-16.
Papkovskii, D. B., A. P. Savitskii, et al. (1990). “Photodestruction in vitro of tumour cells sensitized by porphyrins and their conjugates with specific antibodies.” Biomed Sci 1(4): 401-6.
Papkovsky, D. B. (2004). “Methods in optical oxygen sensing: Protocols and critical analyses.” Methods in Enzymology 381: 715-735.
Papkovsky, D. B. and G. V. Ponomarev (1998). Luminescent dye comprising metallocomplex of a oxoporphyrin. USA, U.S. Pat. No. 5,718,842.
Ponomarev G. V., Yashunsky D. V., Meltola N., Soini A. E., Porphyrin compounds, their conjugates and assay methods based on the use of said conjugates. U.S. Pat. No. 6,582,930, 2003.
Rumsey, W. L., J. M. Vanderkooi, et al. (1988). “Imaging of phosphorescence: a novel method for measuring oxygen distribution in perfused tissue.” Science 241(4873): 1649-51.
Schmalzlin, E., J. T. van Dongen, et al. (2005). “An optical multifrequency phase-modulation method using microbeads for measuring intracellular oxygen concentrations in plants.” Biophysical Journal 89(2): 1339-1345.
Vinogradov, S. A., L. W. Lo, et al. (1996). “Noninvasive imaging of the distribution in oxygen in tissue in vivo using near-infrared phosphors.” Biophys J 70(4): 1609-17.
Vinogradov, S. A. and D. F. Wilson (1998). Phosphorescent dendritic macromolecular compounds for imaging tissue oxygen. U.S. Pat. No. 5,837,865.
Wilson, D. F. and S. A. Vinogradov (2002). Method and apparatus for determining the effect of a drug on cells. U.S. Pat. No. 6,395,555.
Xue, H. H., D. M. Zhao, et al. (2000). “Store depletion by caffeine/ryanodine activates capacitative Ca(2+) entry in nonexcitable A549 cells.” J. Biochem (Tokyo) 128(2): 329-36.

1. A probe for sensing and imaging of intracellular oxygen comprising:
an oxygen-sensitive fluorescent or phosphorescent dye linked to a macromolecular carrier; and
a cell loading component or agent.

2. A probe as claimed in claim 1 wherein the macromolecular carrier and the cell loading component are the same entity.

3. A probe as claimed in claim 1 wherein the dye is conjugated to the macromolecular carrier.

4. A probe as claimed in claim 1 wherein the dye, macromolecular carrier and cell loading agent are combined in one supramolecular structure.

5. A probe as claimed in claim 1 comprising a highly photostable dye suitable for live-cell fluorescence microscopy measurements

6. A probe as claimed in claim 1 wherein the oxygen sensitive dye is selected from the following group: phosphorescent platinum(II) or palladium(II) porphyrins, fluorescent complexes of ruthenium(II) or osmium(II), close analogs or derivatives of these dyes.

7. A probe as claimed in claim 1 wherein the oxygen-sensitive dye comprises Pt-coproporphyrin or a monofunctional reactive derivative thereof conjugated to the macromolecular carrier.

8. A probe as claimed in claim 1 wherein the oxygen-sensitive dye comprises Pt-coproporphyrin-ketone, Pd-coproporphyrin-ketone, or a derivative or close analog thereof.

9. A probe as claimed in claim 1 which is based on a stable Pd-chlorin or Pt-chlorin dye.

10. A probe as claimed in claim 1 which contains two or more oxygen-sensitive dyes with different sensitivities to oxygen.

11. A probe as claimed in claim 1 in which the carrier is a biocompatible, hydrophilic macromolecule.

12. A probe as claimed in claim 1 in which the carrier has molecular weight in the region of 5,000-200,000 D.

13. A probe as claimed in claim 1 in which the carrier is a polypeptide, a polynucleotide, a polysaccharide or a synthetic polymer such as poly(acrylate) or poly(ethyleneglycol).

14. A probe as claimed in claim 13 wherein the carrier comprises an inert protein such as serum albumin, or an antibody or a fragment thereof.

15. A probe as claimed in claim 13 wherein the polypeptide is a cellular targeting polypeptide.

16. A probe as claimed in claim 15 wherein the polypeptide targets the probe to a cellular structure selected from the group comprising: cell surface structures, mitochondria, inner cell membrane(s), lysozymes and/or endoplasmic reticulum.

17. A probe as claimed in claim 3 wherein the macromolecular conjugate at physiological conditions has a net negative charge.

18. A probe as claimed in claim 1 wherein the loading agent comprises a cationic liposomal or non-liposomal transfection agent.

19. A probe as claimed in claim 1 further comprising a protein-free biological buffer as a medium.

20. A probe as claimed in claim 1 wherein the loading agent comprises an endocytosis stimulating agent.

21. Use of a probe as claimed in claim 1 for sensing intracellular oxygen.

22. A method for sensing intracellular oxygen comprising the steps of:
preparing a probe stock solution of desired concentration from a probe as claimed in claim 1;
preparing a sample containing live test cells in a medium compatible with the probe;
adding the probe stock to the sample containing the cells to achieve probe loading;
analyzing fluorescence or phosphorescence from the cells loaded with the oxygen probe on a suitable detector; and
assessing cellular oxygen on the basis of fluorescent or phosphorescent measurements.

23. A method as claimed in claim 22 in which loading of the cells is performed at probe concentration in extracellular space in the region of 10 nM-100 μM (with respect to the dye).

24. A method as claimed in claim 22 in which incubation time for loading the cells with the probe is in the region of 0.2-24 h.

25. A method as claimed in claim 22 in which the cells are loaded with the probe at constant temperature of 30-37° C.

26. A method as claimed in claim 22 in which, after loading the cells and prior to fluorescence/phosphorescence measurement, the probe in the extracellular space is removed by washing the cells.

27. A method as claimed in claim 22 in which fluorescence/phosphorescence intensity signal from the intracellular probe is monitored and related to oxygen concentration.

28. A method as claimed in claim 22 in which fluorescence/phosphorescence intensity signal from the intracellular oxygen probe is monitored in time-resolved mode.

29. A method as claimed in claim 22 in which fluorescence/phosphorescence lifetime of the intracellular oxygen probe is monitored and related to oxygen concentration.

30. A method as claimed in claim 22 in which fluorescence/phosphorescence is monitored kinetically over a period of time.

31. A method as claimed in claim 22 in which fluorescence/phosphorescence measurements with the cells loaded with the probe are performed in a microscopic volume.

32. A method as claimed in claim 22 in which fluorescence/phosphorescence measurements with the cells loaded with the probe are performed on a fluorescent microscope.

33. A method as claimed in claim 32, in which FLIM microscopy is used.

34. A method as claimed in claim 32, in which a fluorescent microscope equipped with a pulsed laser or LED excitation and a fast gated CCD camera is used in time-resolved mode.

35. A method as claimed in claim 32, in which a confocal or a multi-photon excitation fluorescent microscope is used.

36. A method as claimed in claim 32 in which individual cells are monitored.

37. A method as claimed in claim 22 in which fluorescence/phosphorescence measurements with the cells loaded with the probe are performed in a macroscopic volume.

38. A method as claimed in claim 22 in which fluorescence/phosphorescence measurements with the cells loaded with the probe are performed on a fluorescent spectrometer or reader.

39. A method as claimed in claim 22 in which the cells comprise a line of suspension of adherent cells, primary cells or tissue.

40. A method as claimed in claim 22 in which the cells are pre-treated with effector compounds and their effects on cellular oxygen are examined.

41. A method as claimed in claim 22 in which an effector compound is added to the cells during the optical measurements, and alterations in probe signal caused by the effector are monitored and related to cellular oxygen or oxygen consumption.

42. A method as claimed in claim 22 in which fluorescent or phosphorescent measurements are localised to a specific cellular compartment, to which the probe is delivered.

43. A method of preparing a cellular probe comprising the steps of:
conjugating an oxygen-sensitive fluorescent or phosphorescent dye to a hydrophilic macromolecular carrier;
purifying the conjugate; and optionally
adding a cell loading agent to the conjugate.
